SEQUENCE LISTING (5 sequences)

SEQ ID NO: 1 - DNA, 1406 bp, Lactobacillus pentosus
  source 1..1406: mol_type = genomic DNA
cttaggcggctggttcctaaaaggttaccccaccgactttgggtgttacaaactctcatggtgtgacgggcggtgtgtacaaggcccgggaacgtattcaccgcggcatgctgatccgcgattactagcgattccgacttcatgtaggcgagttgcagcctacaatccgaactgagaatggctttaagagattagcttactctcgcgagttcgcaactcgttgtaccatccattgtagcacgtgtgtagcccaggtcataaggggcatgatgatttgacgtcatccccaccttcctccggtttgtcaccggcagtctcaccagagtgcccaacttaatgctggcaactgataataagggttgcgctcgttgcgggacttaacccaacatctcacgacacgagctgacgacaaccatgcaccacctgtatccatgtccccgaagggaacgtctaatctcttagatttgcatagtatgtcaagacctggtaaggttcttcgcgtagcttcgaattaaaccacatgctccaccgcttgtgcgggcccccgtcaattcctttgagtttcagccttgcggccgtactccccaggcggaatgcttaatgcgttagctgcagcactgaagggcggaaaccctccaacacttagcattcatcgtttacggtatggactaccagggtatctaatcctgtttgctacccatactttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaaaaaaccgcctgcgctcgctttacgcccaataaatccggacaacgcttgccacctacgtattaccgcggctgctggcacgtagttagccgtggctttctggttaaataccgtcaatacctgaacagttactctcagatatgttcttctttaacaacagagttttacgagccgaaacccttcttcactcacgcggcgttgctccatcagactttcgtccattgtggaagattccctactgctgcctcccgtaggagtttgggccgtgtctcagtcccaatgtggccgattaccctctcaggtcggctacgtatcattgccatggtgagccgttaccccaccatctagctaatacgccgcgggaccatccagaagtgatagccgaagccatctttcaaactcggaccatgcggtccaagttgttatgcggtattagcatctgtttccaggtgttatcccccgcttctgggcaggtttcccacgtgttactcaccagttcgccactcactcaaatgtaaatcatgatgcaa

SEQ ID NO: 2 - DNA, 20 bp, synthetic construct
  misc_feature 1..20: note = Primer 8F
  source 1..20: mol_type = other DNA
agagtttgatcctggctcag

SEQ ID NO: 3 - DNA, 20 bp, synthetic construct
  misc_feature 1..20: note = Primer 1525R
  source 1..20: mol_type = other DNA
aaggaggtgatccagccgca

SEQ ID NO: 4 - DNA, 1425 bp, Lactobacillus plantarum
  source 1..1425: mol_type = genomic DNA
ggttcctaaaaggttaccccaccgactttgggtgttacaaactctcatggtgtgacgggcggtgtgtacaaggcccgggaacgtattcaccgcggcatgctgatccgcgattactagcgattccgacttcatgtaggcgagttgcagcctacaatccgaactgagaatggctttaagagattagcttactctcgcgagttcgcaactcgttgtaccatccattgtagcacgtgtgtagcccaggtcataaggggcatgatgatttgacgtcatccccaccttcctccggtttgtcaccggcagtctcaccagagtgcccaacttaatgctggcaactgataataagggttgcgctcgttgcgggacttaacccaacatctcacgacacgagctgacgacaaccatgcaccacctgtatccatgtccccgaagggaacgtctaatctcttagatttgcatagtatgtcaagacctggtaaggttcttcgcgtagcttcgaattaaaccacatgctccaccgcttgtgcgggcccccgtcaattcctttgagtttcagccttgcggccgtactccccaggcggaatgcttaatgcgttagctgcagcactgaagggcggaaaccctccaacacttagcattcatcgtttacggtatggactaccagggtatctaatcctgtttgctacccatactttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaaaaaaccgcctgcgctcgctttacgcccaataaatccggacaacgcttgccacctacgtattaccgcggctgctggcacgtagttagccgtggctttctggttaaataccgtcaatacctgaacagttactctcagatatgttcttctttaacaacagagttttacgagccgaaacccttcttcactcacgcggcgttgctccatcagactttcgtccattgtggaagattccctactgctgcctcccgtaggagtttgggccgtgtctcagtcccaatgtggccgattaccctctcaggtcggctacgtatcattgccatggtgagccgttaccccaccatctagctaatacgccgcgggaccatccaaaagtgatagccgaagccatctttcaagctcggaccatgcggtccaagttgttatgcggtattagcatctgtttccaggtgttatcccccgcttctgggcaggtttcccacgtgttactcaccagttcgccactcactcaaatgtaaatcatgatgcaagcaccaatcaataccagagttcgttcgact

SEQ ID NO: 5 - DNA, 1403 bp, Lactobacillus rhamnosus
  source 1..1403: mol_type = genomic DNA
gtcgacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccgg

(19) United States
(12) Patent Application Publication
(10) Pub. No.: US 2024/0342228 A1
(43) Pub. Date: Oct. 17, 2024

(54) SPRAYABLE FORMULATION COMPRISING VIABLE AND/OR STABLE BACTERIA

(71) Applicant: Yun NV, Niel (BE)

(72) Inventors: Tim Henkens, Niel (BE); Ingmar Claes, Niel (BE); Alix Simons, Niel (BE); Imane Gamgami, Niel (BE); Sarah Lebeer, Antwerpen (BE); Ilke De Boeck, Antwerpen (BE); Irina Spacova, Antwerpen (BE)

(73) Assignees: Yun NV, Niel (BE); Universiteit Antwerpen, Antwerpen (BE)

(21) Appl. No.: 18/682,588

(22) PCT Filed: Aug. 12, 2022
(86) PCT No.: PCT/EP2022/072628

§ 371 Date: Feb. 9, 2024

(30) Foreign Application Priority Data
Aug. 12, 2021 (BE) 20215643

(51) Int. Cl. A61K 35/747; A61K 9/10; A61K 47/02; A61K 47/44; A61P 31/14

(52) U.S. Cl. CPC A61K 35/747; A61K 9/10; A61K 47/02; A61K 47/44; A61P 31/14

(57) ABSTRACT

The present invention relates to a sprayable formulation comprising live and/or stable bacteria and methods for making such sprayable formulation; in particular in the form of a liquid spray. The invention further relates to the use of this sprayable formulation in human or veterinarian medicine and the use of this sprayable formulation for the prevention and/or treatment of respiratory diseases. The invention further provides methods for the prevention and/or treatment of respiratory diseases.

### FIELD OF THE INVENTION

The present invention relates to a sprayable formulation comprising live and/or stable bacteria and methods for making such sprayable formulation; in particular in the form of a liquid spray. The invention further relates to the use of this sprayable formulation in human or veterinarian medicine and the use of this sprayable formulation for the prevention and/or treatment of respiratory diseases. The invention further provides methods for the prevention and/or treatment of respiratory diseases.

### BACKGROUND TO THE INVENTION

Respiratory viral infections (influenza, respiratory syncytial virus (RSV), coronaviruses) are linked with a significant health and economic burden, as recently highlighted by the severe acute respiratory syndrome coronavirus 2 (SARS-CoV-2) pandemic. Respiratory tract infections are generally initiated by inhaled viruses infecting the mucosal surfaces of the upper respiratory tract (URT), which includes the nasal cavity, nasopharynx and oropharynx. Viral infections start by virions binding a specific host cell receptor and then invading the epithelial or innate immune cells, depending on the tissue tropism. Subsequent viral replication and host cell damage triggers the activation of first the innate and subsequently the adaptive immune system. Typical transcriptional molecular markers of antiviral innate immune activation are the interferon regulatory factors (IRFs) and nuclear factor kB (NF-kβ), which activate pathways leading to production of type I and Ill interferons and recruitment and activation of leukocytes such as natural killer cells and neutrophils via chemokine production. Excessive or imbalanced immune activation is associated with airway tissue disruption and severe inflammation, as described for the coronavirus disease 2019 (COVID-19). This is characterized by reduced type I and III interferon production occurring together with overproduction of pro-inflammatory mediators such as IL−. An immune overreaction with excessive pro-inflammatory cytokine release recognized as a cytokine storm also occurs in other respiratory viral diseases, such as influenza.

The microbiome that is present at these mucosal surfaces has important multifactorial gatekeeper functions by blocking incoming pathogens and maintaining epithelial barrier function and immune homeostasis. After the initial infection at the URT, several viruses can trigger subsequent pneumonia such as influenza viruses, RSV, human parainfluenza virus (HPIV) and several coronaviruses such as endemic human coronaviruses (eg HCoV-229E) and the recently emerged SARS-CoV-2 and the associated co-infections. Pathobionts including Streptococcus pneumoniae, Staphylococcus aureus, Haemophilus influenzae and Moraxella catarrhalis can potentially benefit from the virus-induced damage and immune dysfunction. Previous studies suggest that the stability of the core URT microbiome is compromised during respiratory viral infection, for instance with influenza, which can pave the way for pathobiont overgrowth. However, it is not yet well studied whether in a significant fraction of severely ill patients the microbiome might not be insufficiently protective against co-infections or the development of severe inflammation.

While the respiratory microbiome is suggested to serve an important gatekeeper function in the airways, few trials up to now have explored respiratory microbiome modulation as a strategy against respiratory viral infections.

Because the ease of formulating probiotics in dry formats such as powders, capsules and tablets, the oral intake of microbiome therapeutics remains the most common administration route today. These oral applications could result in systemic antiviral and anti-inflammatory effects when arriving in the gut through the gut-lung axis. Yet, topical microbiome therapy administration in the URT is increasingly explored as a safe and more targeted alternative to the oral route. Microbiome therapeutics administered to the URT could directly block or inhibit respiratory viruses (prophylaxis), and ensure efficient and direct immune modulation at the site of infection and inflammation. Indeed, targeted local mucosal immune stimulation within the respiratory immune system could have specific benefits compared to the gut-lung axis, for example through more direct and efficient contact with specific cells of the URT immune system, e.g. more efficient induction of regulatory T cells subsets.

Targeted application and delivery of these formulations face some complex technical and biotechnological issues. One of which is that the selected strains need to be sufficiently robust to survive the bioprocessing steps of fermentation for biomass generation, concentration of the biomass and drying, whereby each step needs to be optimized to generate sufficient yield and make the industrial scale process economically interesting without loss of quality. Different drying techniques and processes can be applied to achieve a stable, long term-viability and quality probiotic product, each having their own advantages and disadvantages to consider. The process type, e.g. batch vs continuous, the manufacturing costs, and processing conditions and stresses are important to consider when producing a certain strain.

However, the most important outcome parameter is the applicability of the dried probiotics and the power characteristics such as particle size distribution, density, powder flowability, and surface conditions. These properties determine further processing into targeted URT application formulations. Specifically, a targeted throat application can be achieved via lozenges or a melting tablet with bioadhesives or mucoadhesives such as hydroxypropylcellulose (HPC), carboxymethyl cellulose (CMC), chitosan and different swelling polymers to increase throat retention time. These formulations can be achieved with relative ease due to the extensive research on dry and powdered probiotic formulations such as powders, capsules, tablets and similar derivates. A large disadvantage of these formulations is that they are dry and the microbes inside them need liquid and a certain retention time to resuscitate and re-activate their metabolism when arriving in the throat or oronasopharynx target region. Also, dry powder formulations tend to irritate the throat and oronasopharynx.

A problem is however that dry formulations are not able to specifically target the URT in order to provide protection and/or treatment of respiratory diseases. Also, dry powder formulations tend to irritate the throat and oronasopharynx.

It is therefore an object of this invention to provide a formulation which enables the delivery of microbes in the throat or oronasopharynx target region with a high dispersity and retention time.

It was surprisingly found that a sprayable formulation comprising powdered viable and/or stable bacteria particles suspended in an anhydrous carrier, wherein at least 90% of the powdered bacteria particles have a particle size of less than 400 μm, wherein the sprayable formulation is non-pressurized provides a solution to the above-mentioned problems. In particular, it was found that such a sprayable formulation is easy to use and deposits homogeneously in the throat in contrast to dry formulations. Furthermore, because at least 90% of the powdered bacteria have a particle size of less than 400 μm, the formulation containing the powdered bacteria is sprayable. Moreover, the particle size of the powdered bacteria also allows that the bacteria become widely dispersed when the sprayable formulation of this invention is administered. Said particle size of the powdered bacteria is considered relatively small and therefore causes that the particles have a large contact surface compared particles having a larger particle size. Also due said particle size, there are more particles present per volume compared to larger particle sizes in the same volume, hence more powdered bacteria are deposited in the URT and less particles are removed from the URT by swallowing.

Furthermore, less sedimentation occurs in the suspension when at least 90% of the powdered bacteria has a particle size of less than 400 μm. Therefore, the powdered bacteria are homogeneously present in the suspension.

It was further found that the sprayable formulation of this invention enhances the natural protective function of the oronasopharynx microbiome in respiratory viral disease. In particular, microbiome strains belonging to the Lactobacillaceae show a multifactorial action against the different phases of viral URT infections.

### SUMMARY OF THE INVENTION

In a first aspect, the invention provides a sprayable formulation comprising powdered viable and/or stable bacteria particles suspended in an anhydrous carrier, wherein at least 90% of the powdered bacteria particles have a particle size of less than 400 μm, wherein the sprayable formulation is non-pressurized.

In particular, the invention provides a sprayable formulation comprising powdered viable non-sporulating bacteria particles suspended in a non-volatile anhydrous liquid carrier, wherein at least 90% of the powdered bacteria particles have a particle size of less than 400 μm, wherein the sprayable formulation is under ambient pressure; in particular wherein the sprayable formulation is in the form of a liquid spray.

In a further embodiment, the sprayable formulation does not comprise propellant gas, in particular carbohydrate propellant gas.

In another embodiment, the sprayable formulation is dispersed in droplets when sprayed.

In a particular embodiment of the invention, the anhydrous carrier (in particular the non-volatile anhydrous liquid carrier) of the sprayable formulation has a vegetable origin, animal origin or mineral origin, preferably wherein the anhydrous carrier (in particular the non-volatile anhydrous liquid carrier) is an oily liquid carrier, more preferably comprising fatty acids, triglycerides, saturated or unsaturated fats, steroid derivatives or complex oils composing of phospholipids, sphingolipids, glycolipids or sulpholipids.

In another embodiment of the invention, at least 90% of the powdered bacteria particles have a particle size of less than 400 μm, preferably between 1 and 250 μm, more preferably between 2 and 100 μm, even more preferably between 5 and 50 μm, most preferably between 5 and 10 μm.

In a particular embodiment of the present invention, the concentration of powdered bacteria particles in the suspension is between 0.1-20 wt. %, preferably between 1-10 wt. %, most preferably between 3 and 7 wt. % in respect to the total weight of the sprayable formulation.

In another particular embodiment the sprayable formulation further comprises an anti-sedimentation agent and in yet a further particular embodiment the anti-sedimentation agent is silicon dioxide and derivatives thereof.

In a particular embodiment of the present invention the concentration of the anti-sedimentation agent is 0.01-10 wt. %, preferably between 0.1-5 wt. %, more preferably between 0.5-3 wt. % and most preferably 1-2.5 wt. % in respect to the total weight of the sprayable formulation.

In another particular embodiment of the present invention, the sprayable formulation comprises between 50-99 wt. % anhydrous carrier, preferably between 70-98 wt. %, more preferably between 85 and 97 wt. %, most preferably between 90 and 95 wt. % in respect to the total weight of the sprayable formulation.

In yet a further embodiment of the present invention, the sprayable formulation comprises between 50-99 wt. % non-volatile anhydrous liquid carrier, preferably between 70-98 wt. %, more preferably between 85 and 97 wt. %, most preferably between 90 and 95 wt. % in respect to the total weight of the sprayable formulation.

In a specific embodiment of the present invention, the sprayable formulation further comprises a rheological additive agent.

In a further specific embodiment of the present invention, the sprayable formulation further comprises antioxidants, in particular selected from vitamin D3 and E.

In another specific embodiment of the present invention the sprayable formulation further comprises surfactants and/or emulsifiers and/or humectants.

In a particular embodiment of the present invention, the sprayable formulation further comprises bio-adhesives and/or mucoadhesives.

In another particular embodiment the sprayable formulation of the present invention further comprises sweeteners and/or flavors.

In a particular embodiment the sprayable formulation of the present invention further comprises formulation stabilizers in particular selected from the group consisting of epicatechins, quinones, creatin, hydroxytyrosol, pyridoxamine, cysteine, homocysteine, gluthation or other trapping alfa carbonyls.

In a further particular embodiment the viable and/or stable bacteria in the sprayable formulation of the present invention are probiotic bacteria, in particular lactic acid bacteria or Staphylococcus species; more in particular Lactobacillus species.

In another particular embodiment the lactic acid bacteria in the sprayable formulation of the present invention are Streptococcus species or Lactobacillus species such as L. plantarum, L. pentosus, L. rhamnosus and/or L. casei.

In another specific embodiment the sprayable formulation is an oronasopharyngeal spray.

In a further specific embodiment the sprayable formulation of the present invention is a topical dermatological spray.

In a further particular embodiment, the invention relates to the sprayable formulation of the present invention for use in human or veterinarian medicine.

In another particular embodiment, the present invention relates to the sprayable formulation of the present invention for use in enhancing the natural protective function of the oronasopharynx microbiome.

In a further particular embodiment, the invention relates to the sprayable formulation of the present invention for use in the prevention and/or treatment of viral, bacterial and/or fungal respiratory diseases.

In another particular embodiment, the invention relates to the sprayable formulation of the present invention for use in the prevention and/or treatment of coronaviral diseases.

In a further aspect, the present invention provides a method for preparing the sprayable formulation of the present invention, comprising the step of suspending powdered bacteria particles in an anhydrous carrier; more in particular in a non-volatile anhydrous liquid carrier.

In a further embodiment, the present invention relates to a method for enhancing the natural protective function of the oronasopharynx microbiome, comprising the step of applying the sprayable formulation of this invention.

In yet a further embodiment, the present invention provides a method for preventing and/or treating viral, bacterial and/or fungal respiratory diseases, comprising the step of applying the sprayable formulation of the present invention.

In a further particular embodiment, the recent invention provides a method for preventing and/or treating coronaviral diseases, comprising the step of applying the sprayable formulation of the present invention.

In another particular embodiment, the invention relates to the use of the sprayable formulation of the present invention for a topical dermatological application.

In a further particular embodiment, the invention relates to the use of the sprayable formulation of the present invention for cleaning surfaces.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Time course experiment of the viability of different bacteria concentration in oil with 2% silicon dioxide at 15° C. expressed in CFU/g oil suspension. The control is a probiotic mixture of L. rhamnosus and L. plantarum which is expressed as CFU/gram powder (pure strain not diluted in the oil carrier).

FIG. 2: Time course experiment of the viability of different bacteria concentration in oil with 2% silicon dioxide at 25° C. expressed in CFU/g oil suspension. The control is a probiotic mixture of L. rhamnosus and L. plantarum which is expressed as CFU/gram powder (pure strain not diluted in the oil carrier).

FIG. 3: Retention of lactobacilli in the throat after 30 min, 2 and 4 hours after application. The presence of lactobacilli in the throat is tested on 3 different persons, denoted as P1-P2-P3, by swabbing the soft palate and the back end of the tongue (surface of ±2.5 cm²).

FIG. 4: Viability data of different bacteria concentration in oil with 2% silicon dioxide at 4° C.

FIG. 5: Viability of individual and combined L. casei AMBR2, L. plantarum WCFS1 and L. rhamnosus GG in (A-B) powder or (C) the throat spray formulation; Immunostimulatory activity of the powders (D-E) and the placebo and verum spray formulation without or with lactobacilli, respectively (F-G). The viability of the strains combination was evaluated at different storage temperatures over time; 4° C., 15° C. and 25° C. CFU: colony-forming units. The medium condition represents the cells as such and serves as a baseline, Poly(I:C) at 50 μg/ml with Lipofectamine (Poly(I:C)50/Lipo) serves as control IRF inducer and LPS at 20 ng/ml (LPS20) serves as control NF-κ B inducer. Data is depicted as mean±SD per condition. *p<0.05, **p<001 ***p<0.001 and ****p<0.0001 as determined by a One-way ANOVA test followed by Dunnett's multiple comparisons test compared to the medium condition (dotted line).

FIG. 6: Evaluation of lactobacilli retention within the throat microbiome of healthy volunteers after spray application. (A) Study set-up; (B) Relative abundance of the administered lactobacilli in throat swabs based on microbiome analysis via 16S rRNA amplicon sequencing; (C) Relative abundance of the administered lactobacilli in throat swabs based on qPCR analysis. Throat swabs were collected at baseline (TO), and 30 minutes (T1) and 2 hours (T2) after the throat spray was used. The presence of L. casei AMBR2, L. rhamnosus GG, and L. plantarum WCFS1 was evaluated via 16S rRNA amplicon sequencing (relative abundances) in panel B. At 30 minutes and 2 hours, qPCR with species-specific primers was used to estimate the CFU/ml counts in the verum group in panel D. Based on the standard curve, the detection limit was estimated to be at 10³ CFU/ml.

### DETAILED DESCRIPTION OF THE INVENTION

As already detailed herein above, the present invention provides a sprayable formulation comprising powdered viable and/or stable bacteria particles suspended in an anhydrous carrier; more in particular in a non-volatile anhydrous liquid carrier.

In particular, the present invention provides a sprayable formulation comprising powdered viable and/or stable bacteria particles suspended in an anhydrous carrier, wherein at least 90% of the powdered bacteria particles have a particle size of less than 400 μm, wherein the sprayable formulation is non-pressurized.

In another embodiment, the present invention provides a sprayable formulation comprising powdered viable non-sporulating bacteria particles suspended in a non-volatile anhydrous liquid carrier, wherein at least 90% of the powdered bacteria particles have a particle size of less than 400 μm, wherein the sprayable formulation is under ambient pressure, and wherein the sprayable formulation is in the form of a liquid spray.

In a further embodiment, the sprayable formulation does not comprise propellant gas, in particular carbohydrate propellant gas.

In another embodiment, the sprayable formulation is dispersed in droplets when sprayed.

In a particular embodiment of the invention, the anhydrous carrier (more in particular the non-volatile anhydrous liquid carrier) of the sprayable formulation has a vegetable origin, animal origin or mineral origin, preferably wherein the anhydrous carrier (in particular the non-volatile anhydrous liquid carrier) is an oily liquid carrier, more preferably wherein the non-volatile anhydrous liquid carrier comprises fatty acids, triglycerides, saturated or unsaturated fats, steroid derivatives or complex oils composing of phospholipids, sphingolipids, glycolipids or sulpholipids.

In a further particular embodiment of the invention, at least 90% of the powdered bacteria particles have a particle size between 1 and 250 μm, more preferably between 2 and 100 μm, most preferably between 5 and 50 μm.

In another embodiment of the present invention, at least 90% of the powdered bacteria have a particle size of less than 400 μm, preferably between 1 and 250 μm, more preferably between 2 and 100 μm, even more preferably between 5 and 50 μm, most preferably between 5 and 10 μm.

In a particular embodiment at least 99% of the powdered bacteria have a particle size of less than 50 μm, and/or at least 80% of the powdered bacteria have a particle size of less than at least 30 μm and/or 35% of the powdered bacteria have a particle size of less than 10 μm.

In the context of the present invention, the term “sprayable” formulation should be interpreted as a combination of components being able to move in a mass of dispersed droplets. A sprayable formulation can further be a non-pressurized formulation or a pressurized formulation such as an aerosol sprayable formulation or a sprayable formulation suitable for a bag-on-valve system.

Accordingly, where in the application, reference is made to a ‘sprayable formulation’ this is meant to be a ‘liquid spray’, i.e. a liquid composition which is applied by spraying said composition. A ‘liquid spray’ as used herein is a liquid formulation or composition that is blown or driven through the air, or forced out of a holder, in the form of a mist or tiny drops or droplets.

In the context of the present invention the term microorganism refers to “viable” bacteria particles, meaning that the bacteria are alive, and it is not meant to be fragments, culture supernatants, fermented forms, or killed forms thereof. Said viable bacteria particles are preferably freeze-dried or spray-dried in order to increase their preservation.

In the context of the present invention, the term “powdered” bacteria refers to that the bacteria particles are fine dry particles, such as in the form of a powder or fine dust.

Further, in the context of the present invention the term microorganism refers to “stable” bacteria particles, which means that the particles which contain the viable bacteria are stable when they are suspended in an anhydrous carrier (in particular a non-volatile anhydrous liquid carrier). In particular, the microorganisms of the present invention are preferably in a dormant state, such that they do not replicate/multiply in the sprayable formulation itself. With ‘stable’ bacteria it is meant that the microorganisms remain in that ‘dormant’ state and will only be activated upon application of the sprayable formulation.

Also in the context of the present invention the term “particle size” refers to the average intersection or diameter of all of the bacteria particles. Specifically, it means that the majority of particles in the formulation have a diameter within the specified ranges. Specifically, at least 50%, at least 60%, at least 70%, at least 75%, at least 80%, at least 85%, at least 90% or at least 95% of the particles in the formulation have a diameter within the specified ranges.

The powdered viable and/or stable bacteria particles can comprise probiotic bacteria. In the context of the present invention, the term “probiotic” is meant to include bacteria that provide health benefits when used in the human or veterinary field. The formulations of the present invention are highly suitable in the formulation of any known probiotic microorganisms, such as but not limited to Lactobacilli, more in particular Lactobacillus pentosus, Lactobacillus rhamnosus, Lactobacillus plantarum and/or Lactobacillus casei. Evidently, the formulations of the present invention may comprise only one species of probiotic microorganisms, or combinations thereof, depending on the intended use.

In a particular embodiment, the present invention provides a sprayable formulation as defined herein, wherein the concentration of powdered bacteria particles in the suspension is between 0.1-20 wt. %, preferably between 1-10 wt. %, most preferably between 3 and 7 wt. % in respect to the total weight of the sprayable formulation.

More specifically, the present invention provides a sprayable formulation as defined herein, wherein the formulation further comprises an anti-sedimentation agent. These anti-sedimentation agents can be silicon dioxide and derivatives thereof, Furthermore, the anti-sedimentation agent can be hydrophilic or hydrophobic. Additionally, the anti-sedimentation agents can be bentonite, or anti-sedimentation agents based on electrolytes. The anti-sedimentation agents are added to avoid sedimentation of the powdered bacterial particles suspended in the anhydrous carrier (in particular the non-volatile anhydrous liquid carrier).

The present invention further provides that the anti-sedimentation agent is present in 0.01-10 wt. %, preferably between 0.1-5 wt. %, more preferably between 0.5-3 wt. % and most preferably 1-2.5 wt. % in respect to the total weight of the sprayable formulation. In particular, after 3 months no sedimentation of the powdered bacterial particles suspended in the anhydrous carrier (in particular the non-volatile anhydrous liquid carrier) is visible when 1-2.5% silicon dioxide was present in the sprayable formulation as defined herein.

In a further particular embodiment, the sprayable formulation of the present invention comprises between 50-99 wt. % anhydrous carrier (in particular the non-volatile anhydrous liquid carrier), preferably between 70-98 wt. %, more preferably between 85 and 97 wt. %, most preferably between 90 and 95 wt. % in respect to the total weight of the sprayable formulation.

In the context of this invention the term “anhydrous” carrier means a liquid which substantially does not comprise water, i.e. is substantially free from water. In particular, the anhydrous carrier (in particular the non-volatile anhydrous liquid carrier) may contain a maximum amount of water of less than 5 wt %, in particular less than 4 wt %, less than 3 wt %, less than 1 wt %, less than 0.5 wt % of water in respect to the total weight of the sprayable formulation. A stable suspension can be provided when the powdered viable and/or stable bacteria particles are suspended in the anhydrous carrier (in particular the non-volatile anhydrous liquid carrier).

The present invention further provides a sprayable formulation as defined herein, further comprising a rheological additive agent. Rheological additive agents can be present to alter the viscosity of the sprayable formulation. Typical rheological additive agents which can be present in the sprayable formulation of the invention are polysaccharides, e.g. cellulose derivates, xanthan gum, pectin, and silicon derivates.

Also, the present invention provides a sprayable formulation as defined herein, further comprising antioxidants, in particular selected from vitamin D3 and E. Vitamins or other antioxidants can be present in the sprayable formulation of the invention to provide an antioxidant effect. Peroxides have a negative effect on the functionality of the sprayable formulation as defined herein. Furthermore, oxidation caused by peroxides must be controlled to prevent rancidification of the formulation and deteriorating the formulations flavor profile over time. The antioxidant effect neutralizes the peroxide to enhance the functionality of the sprayable formulation. Further antioxidants can be for example ascorbic acid, sodium bisulfite, sodium thiosulfate, ascorbyl palmitate, butyhydroxyanisol butylhydroxytolune, propyl gallate, alpha-tocopherol, disodium EDTA.

Furthermore, the present invention provides a sprayable formulation as defined herein, wherein the sprayable formulation further comprises surfactants and/or emulsifiers and/or humectants. Typical surfactants/emulsifiers/humectants are for example glycerin, polyols, fatty acid esters.

The present invention further provides a sprayable formulation as defined herein, wherein the sprayable formulation further comprises bio-adhesives and/or mucoadhesives. These adhesives can be present in the sprayable formulation of the invention in order to increase throat retention time. Bio-adhesives and/or mucoadhesives can be for example cellulose derivatives like hydroxypropylcellulose (HPC), carboxymethyl cellulose (CMC), chitosan and/or different swelling polymers like polyacrylic acid.

In a particular embodiment, the present invention provides a sprayable formulation as defined herein, further comprising sweeteners and/or flavors. The flavors and sweeteners which can be present in the sprayable formulation of the present invention are typically Orange A, Orange B, Lemon, Toothpaste, Mint, Peppermint, Cinnamon, Toffee, Caramel, Coffee, Grapefruit, Strawberry/banana, blackberry, cherry, vanilla, raspberry, banana, strawberry.

The present invention further provides a sprayable formulation as defined herein, wherein the formulation further comprises formulation stabilizers in particular selected from the group consisting of epicatechins, quinones, creatin, hydroxytyrosol, pyridoxamine, cysteine, homocysteine, gluthation or other trapping alfa carbonyls. The stabilizers can be present in the sprayable formulation of the invention in order to reduce the Maillard reaction. The Maillard reaction can be a chemical reaction between reducing sugars and amino acids, which are both inherently present in microorganisms. This reaction will have an impact on odor and color stability of the formulation. The formulation stabilizers reduce the occurrence of the Maillard reaction and thereby stabilize the color and odor of the sprayable formulation as defined herein.

In a specific embodiment, the viable and/or stable bacteria of the sprayable formulation of the invention are probiotic bacteria, in particular lactic acid bacteria or Staphylococcus species. The sprayable formulation as defined herein can comprise probiotic lactic acid bacteria (in particular Lactobacillus species) and administration of this sprayable formulation enables modulation of the respiratory microbiome. The probiotic activity of lactic acid bacteria in the sprayable formulation as defined herein, is thus the prevention and/or treatment of respiratory diseases.

More specifically, Streptococcus species and/or Lactobacillus species can be present in the sprayable formulation of the present invention. These species are able to provide the prevention and/or treatment of the respiratory diseases. Even more specifically, the sprayable formulation of the present invention can comprise Lactobacillus species such as L. plantarum, L. pentosus, L. rhamnosus and/or L. casei. Live L. casei, L. rhamnosus and L. plantarum significantly activate the nuclear factor (NF)-kB and interferon regulatory factor (IRF) pathways in human THP-1 Dual monocytes. L. casei and L. plantarum specifically induced expression of interferon-β (IFN-β), a type I interferon essential in antiviral responses, in human primary airway epithelial cells. UV-inactivated L. rhamnosus and L. plantarum maintained their capacity to activate NF-kB and IRF pathways in human THP-1 Dual monocytes. The combination of L. casei, L. rhamnosus and L. plantarum yielded superior induction of NF-kB and IRF pathways in human THP-1 Dual monocytes.

In a particular embodiment, the L. plantarum which can be present in the sprayable formulation of the invention is a L. plantarum strain having at least 97% sequence similarity with SEQ ID N° 4 in its 16S rRNA gene.

In a further particular embodiment, the L. pentosus which can be present in the in the sprayable formulation of the invention is a L. pentosus strain having at least 97% sequence similarity with SEQ ID No 1 in its 16S rRNA gene.

Also, in another particular embodiment, the L. rhamnosus which can be present in the in the sprayable formulation of the invention is a L. rhamnosus strain having at least 97% sequence similarity with SEQ ID No 5 in its 16S rRNA gene.

In particular, the present invention further provides the sprayable formulation as defined herein, wherein the Lactobacillus strain is selected from the list comprising L. pentosus YUN-V1.0 deposited under accession number LMG P-29455 (deposited at BCCM on Mar. 9, 2016); L. plantarum YUN-V2.0 deposited under accession number LMG P-29456 (deposited at BCCM on Mar. 9, 2016); and L. rhamnosus YUN-S1.0 deposited under accession number LMG P-29611 (deposited at BCCM on May 12, 2016).

The microbiological deposits mentioned herein, have been made with the BCCM/LMG Bacteria collection (“Belgian co-ordinated collections of micro-organism”) with correspondence address: Laboratorium voor Microbiologie, Universiteit Gent, K.L. Ledeganckstraat 35-9000 Gent, Belgium

Lactobacillus pentosus YUN-V1.0 is a single colony isolate obtained in our lab after subculturing of a strain, that was originally a vaginal isolate of healthy woman. The 16S rRNA gene sequence (SEQ ID No 1) for strain L. pentosus YUN-V1.0 was determined by PCR using primers 8F (5′-AGAGTTTGATCCTGGCTCAG-3′-SEQ ID No 2) and 1525R (5′-AAGGAGGTGATCCAGCCGCA-3′-SEQ ID No 3).

YUN-V2.0 is a single colony isolates obtained in our lab after subculturing of Lactobacillus plantarum strain that were originally isolated from human saliva. The 16S rRNA gene sequence (SEQ ID No 4) for strain L. plantarum YUN-V2.0 was determined by PCR using primers 8F (5′-AGAGTTTGATCCTGGCTCAG-3′-SEQ ID No 2) and 1525R (5′-AAGGAGGTGATCCAGCCGCA-3′-SEQ ID No 3).

YUN-S1.0 is a single colony isolate obtained in our lab after subculturing of a Lactobacillus rhamnosus strain that was originally isolated from a healthy person. The 16S rRNA gene sequence (SEQ ID No 5) for strain L. rhamnosus YUN-S1.0 was determined by PCR using primers 8F (5′-AGAGTTTGATCCTGGCTCAG-3′-SEQ ID No 2) and 1525R (5′-AAGGAGGTGATCCAGCCGCA-3′-SEQ ID No 3).

These particular “YUN” strains can either be used as such, or are preferably formulated in a composition comprising such strains, in particular a sprayable formulation in the form of a liquid spray.

When administered, the sprayable formulation of the present invention enhances the natural protective function of the oronasopharynx microbiome in respiratory viral disease. The present invention thus further provides a sprayable formulation as defined herein, wherein the sprayable formulation is an oronasopharyngeal spray.

Another aspect of the present invention is that the sprayable formulation can be a topical dermatological spray. The topical application of the sprayable formulation of the present invention can be applied topically to the skin to restore the skin microbiota. In particular, the sprayable formulation can be used to treat symptoms of skin disorders related to overgrowth by pathobionts, for example staphylococci, Malassezia spp., Trichophyton spp. Furthermore, the sprayable formulation of the present invention can be used to reduce malodour production by specific malodour producing skin bacteria.

Also disclosed herein is that the sprayable formulation of the invention can also be an oil suspension. This oil suspension can be applied as topical dermatological oil. Alternatively, this oil suspension can be applied for preventing and/or treating diseases in the outer ear canal such as otitis externa.

A further embodiment of the invention is that the sprayable formulation as described herein can be used in human and/or veterinarian medicine. The sprayable formulation of the present invention can thus be used as a medicament. In particular, the sprayable formulation of the present invention can be used as a medicament, in particular for enhancing the natural protective function of the oronasopharynx microbiome.

In a further particular embodiment, the sprayable formulation of the present invention can be used in the prevention and/or treatment of viral, bacterial and/or fungal respiratory diseases. In a more particular embodiment, the sprayable formulation of the present invention can be used in the prevention and/or treatment of coronaviral diseases. In particular, the sprayable formulation of the present invention is used for preventing and/or treating COVID-19.

In a further aspect, the present invention relates to a method for preparing the sprayable formulation as defined herein, comprising the step of suspending powdered bacteria particles in an anhydrous carrier (in particular the non-volatile anhydrous liquid carrier).

Furthermore and in a further aspect, the present invention relates to a method for enhancing the natural protective function of the oronasopharynx microbiome, comprising the step of applying the sprayable formulation as defined herein.

More particular, the present invention relates to a method for preventing and/or treating viral, bacterial and/or fungal respiratory diseases, comprising the step of applying the sprayable formulation as defined herein.

Also, in particular the present invention relates to a method for preventing and/or treating coronaviral diseases, comprising the step of applying the sprayable formulation as defined herein.

In particular this method is applied for preventing and/or treating COVID-19.

Moreover, and in a further aspect, the present invention relates to the use of a sprayable formulation as defined herein for a topical dermatological application.

Also the present invention relates to the use of sprayable formulation as defined herein for cleaning surfaces.

The invention will now be illustrated by means of the following synthetic and biological examples, which do not limit the scope of the invention in any way.

### EXAMPLES

### Example 1—Particle Size and Sedimentation

TABLE 1
Percentage of sedimentation of bacterial powder (L. rhamnosus) with different
particle size in oil suspensions. Evaluation done at room temperature.

| | | Coarse powder | Fine powder | Very fine powder |
|---|---|---|---|---|
| | | 97% particles below | 100% of particles below | 100% of particles below |
| | | 400 μm, with at least | 400 μm, with at least 90% | 400 μm, with at least 99 |
| | | 25% less than 50 μm | less than 50 μm | % less than 50 μm |
| 15 | min | 10% | 0% | 0% |
| 30 | min | 16% | 8% | 4% |
| 45 | min | 20% | 12% | 4% |
| 1 | hour | 20% | 12% | 4% |

| | | | |
|---|---|---|---|
| 1 hour 30 | 20% | 15% | 8% |
| min | | | |

A 20% sedimentation means that the bacterial powder is completely sedimented. Coarse powder sediments faster than fine and very fine powder. Using fine or very fine powder in an oil suspension are the preferred option to achieve a stable suspension over time.

### Example 2—Effect of Silicon Dioxide as Anti-Sedimentation Agent

This method demonstrates the effect of increasing concentration (1-1.5-2-2.5-3% m/m) of anti sedimentation agent (Silicon dioxide) in probiotic (1-2.5-5-10% m/m) oil suspension of vegetable origin (87-98% m/m). The sedimentation speed, agglomeration (visual control) and probiotic viability are evaluated. The probiotic mixture is a combination of L. rhamnosus YUN-S1.0 and L. plantarum YUN-V2.0.

TABLE 2
Coding system to refer to concentrations of bacteria and silicon dioxide
in the oil suspension. The number refers to the concentration bacteria
and the letter to the concentration of silicon dioxide.

| | | Silicon dioxide | | | | |
|---|---|---|---|---|---|---|
| | | 1% | 1.50% | 2% | 2.50% | 3% |
| bacteria | 1% | 1a | 1b | 1c | 1d | 1e |
| | 2.50% | 2.5a | 2.5b | 2.5c | 2.5d | 2.5e |
| | 5% | 5a | 5b | 5c | 5d | 5e |
| | 10% | 10a | 10b | 10c | 10d | 10e |

The sedimentation and agglomeration is measured in graduated tubes at start, T3d, T1w, T2w, T1m, T2m, T3m, T6m, T12m, T18m, T24m, T36m. Viability of probiotics will be measured at TO, T3m, T6m, T9m, T12m, T18m, T24m, T36m at 4° C.-15° C. & 25° C. via spread plating and colony counting.

Viscosity (in mPa·s) of the suspensions was measured using a Brookfield viscometer DV1 at 22+/−2° C.

TABLE 3
Viscosity of the suspensions

| | | Silicon dioxide | | | | |
|---|---|---|---|---|---|---|
| | | 1% | 1.50% | 2% | 2.50% | 3% |
| bacteria | 1% | 85.6 | 86 | 90.8 | 97.6 | 106.8 |
| | 2.50% | 96 | 90.8 | 96.4 | 99.2 | 107.2 |
| | 5% | 96.8 | 92 | 98 | 108.4 | 120 |
| | 10% | 95 | 102 | 114.4 | 131.2 | 144 |

TABLE 4
Sedimentation data at 4° C.

| Time | | 1a | 1b | 1c | 1d | 1e | 2.5a | 2.5b | 2.5c | 2.5d | 2.5e |
|---|---|---|---|---|---|---|---|---|---|---|---|
| 3 | days | + | + | + | + | 0 | ++ | ++ | + | 0 | 0 |
| 1 | week | + | + | + | + | 0 | ++ | ++ | + | 0 | 0 |
| 2 | weeks | + | + | + | + | 0 | ++ | ++ | + | 0 | 0 |
| 1 | month | + | + | + | + | 0 | ++ | ++ | + | 0 | 0 |
| 2 | months | + | + | + | + | 0 | ++ | ++ | + | + | 0 |
| 3 | months | ++ | + | + | + | 0 | ++ | ++ | + | 0 | 0 |
| 6 | months | ++ | + | + | + | 0 | ++ | ++ | ++ | 0 | 0 |
| 12 | months | ++ | ++ | + | + | 0 | +++ | +++ | ++ | 0 | 0 |
| 18 | months | ++ | ++ | + | + | 0 | +++ | +++ | ++ | 0 | 0 |

| Time | | 5a | 5b | 5c | 5d | 5e | 10a | 10b | 10c | 10d | 10e |
|---|---|---|---|---|---|---|---|---|---|---|---|
| 3 | days | ++ | ++ | + | 0 | 0 | + | 0 | 0 | 0 | 0 |
| 1 | week | ++ | ++ | + | 0 | 0 | + | + | 0 | 0 | 0 |
| 2 | weeks | ++ | ++ | + | 0 | 0 | + | + | 0 | 0 | 0 |
| 1 | month | ++ | ++ | ++ | + | + | ++ | + | + | 0 | 0 |
| 2 | months | ++ | ++ | ++ | + | 0 | +++ | + | + | 0 | 0 |
| 3 | months | ++ | ++ | ++ | 0 | 0 | +++ | ++ | 0 | 0 | 0 |
| 6 | months | +++ | +++ | ++ | 0 | 0 | +++ | +++ | ++ | 0 | 0 |
| 12 | months | +++ | +++ | +++ | 0 | 0 | +++ | +++ | 0 | 0 | 0 |
| 18 | months | +++ | +++ | +++ | 0 | 0 | +++ | +++ | 0 | 0 | 0 |

“0” = no sedimentation,
“+” = between 0 and 5% sedimentation,
“++” = between 5 and 10% sedimentation,
“+++” = >10% sedimentation

TABLE 5
Sedimentation data at 25° C.

| Time | | 1a | 1b | 1c | 1d | 1e | 2.5a | 2.5b | 2.5c | 2.5d | 2.5e |
|---|---|---|---|---|---|---|---|---|---|---|---|
| 3 | days | + | + | + | 0 | 0 | + | + | + | 0 | 0 |
| 1 | week | + | + | + | 0 | 0 | + | +++ | + | 0 | 0 |
| 2 | weeks | + | + | + | + | 0 | + | +++ | + | 0 | 0 |
| 1 | month | + | + | + | + | 0 | + | +++ | + | 0 | 0 |
| 2 | months | + | + | + | 0 | 0 | ++ | +++ | + | 0 | 0 |
| 3 | months | 0 | 0 | 0 | 0 | 0 | 0 | 0 | 0 | 0 | 0 |
| 6 | months | ++ | ++ | + | + | 0 | ++ | ++ | + | 0 | 0 |
| 12 | months | ++ | ++ | + | 0 | 0 | +++ | ++ | + | 0 | 0 |
| 18 | months | ++ | ++ | + | 0 | 0 | ++ | ++ | + | 0 | 0 |

| Time | | 5a | 5b | 5c | 5d | 5e | 10a | 10b | 10c | 10d | 10e |
|---|---|---|---|---|---|---|---|---|---|---|---|
| 3 | days | + | 0 | 0 | 0 | 0 | 0 | 0 | 0 | 0 | 0 |
| 1 | week | ++ | 0 | 0 | 0 | 0 | 0 | 0 | 0 | 0 | 0 |
| 2 | weeks | ++ | 0 | 0 | 0 | 0 | 0 | 0 | 0 | 0 | 0 |
| 1 | month | ++ | 0 | 0 | 0 | 0 | 0 | 0 | 0 | 0 | 0 |
| 2 | months | +++ | 0 | 0 | 0 | 0 | 0 | 0 | 0 | 0 | 0 |
| 3 | months | 0 | 0 | 0 | 0 | 0 | 0 | 0 | 0 | 0 | 0 |
| 6 | months | +++ | 0 | 0 | 0 | 0 | 0 | 0 | 0 | 0 | 0 |
| 12 | months | +++ | 0 | 0 | 0 | 0 | 0 | 0 | 0 | 0 | 0 |
| 18 | months | +++ | 0 | 0 | 0 | 0 | 0 | 0 | 0 | 0 | 0 |

“0” = no sedimentation,
“+” = between 0 and 5% sedimentation,
“++” = between 5 and 10% sedimentation,
“+++” = >10% sedimentation

Surprisingly, it was found that less sedimentation occurred at 25° C. than 4° C. As viscosity increases at lower temperatures, one would expect a slower sedimentation speed of the suspended powder particles. It was also found (at 25° C.) that lower probiotic concentrations (1 and 2.5% m/m) show more sedimentation than the higher concentrations of 5 and 10% whilst the opposite was true at 4° C.

To obtain a stable suspension at room temperature, with this type of silicon dioxide and the selected probiotic powder, a probiotic concentration of 5% with at least 1.5% silicon dioxide is preferable.

At 4° C. and 25° C. no sedimentation was noted for 2.5 and 3% silicon dioxide whilst a slight sedimentation zone was found at 2% silicon dioxide.

2% of this type of hydrophobic modified silicon dioxide seems to be the preferred to achieve a stable suspension over time but spray characteristics need to be taken into account at these higher concentrations due to the increase in viscosity.

### Example 3—Viability

Viability of different bacteria was tested in oil with 2% silicon dioxide at 15° C. and 25° C., wherein the bacteria are present in bacterial powder particles of which at least 90% have a particle size of less than 400 μm.

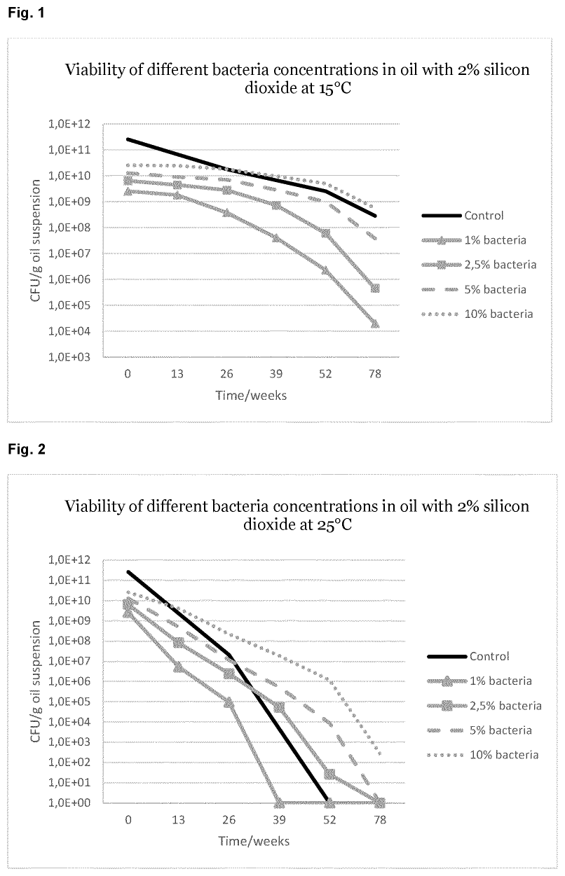

As shown in FIG. 1, the viability was maintained for all formulations when stored at 15° C. At 25° C. (FIG. 2) a faster reduction of viable counts was observed for lower starting concentrations of probiotic bacteria. Therefore, a concentration of 5% (or more) bacteria powder particles in the sprayable formulation appears to be preferred in that it results in a long term stable and viable product.

### Example 4—Throat Retention

Tested sprayable formulation (in the form of a liquid spray):
3 Probiotic strains 5% m/m: L. plantarum YUN-V2.0, L. casei Ambr2, L. rhamnosus YUN-S1.0;
Antioxidant: 0.1% m/m
Vitamin D3 0.01% m/m: 100 IU/gram
Hydrophilic silicon dioxide 1.5% m/m
Vegetable oil carrier 93.4% m/m: mixture of triglycerides and fatty acids

Three persons participated in this investigation.

A blank swab (Copan) was taken from each test person to evaluate the presence of lactobacilli before use of the throat spray. A swab sample was taken from the back end of the soft palate and the back end of the tongue by rubbing the swab for approximately 5 seconds over an area of approximately 2.5 cm².

The tips of the swabs are mixed in 1 ml 0.1 M PBS buffer. A 10-fold serial dilution series is made to determine the CFU count on MRS via the spread plating method. Test persons sprayed ±500 microliters of the sprayable formulation using a suitable spray pump with throat applicator. Samples were collected after 30-120-240 minutes of using the throat spray. No drinking or eating was allowed during this time.

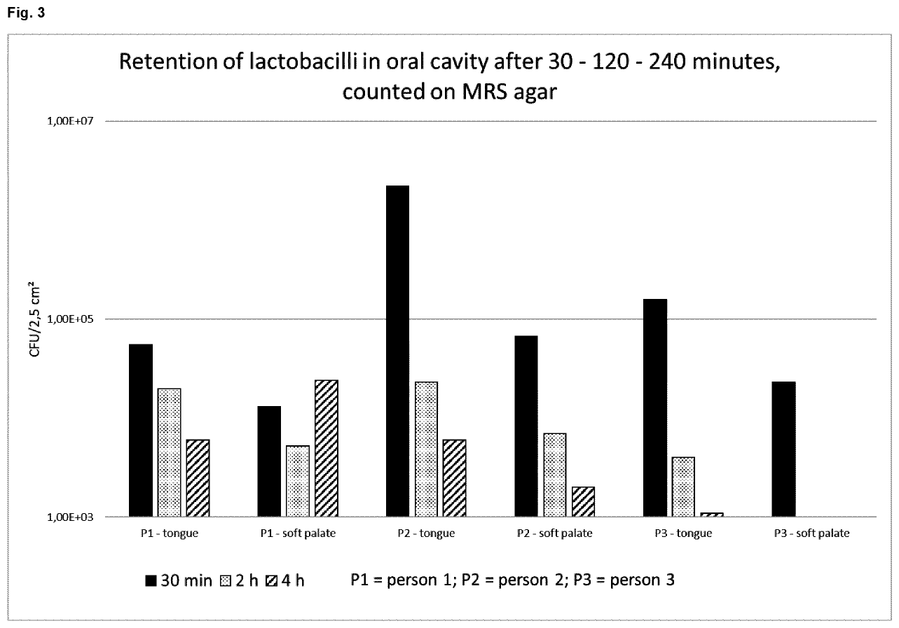

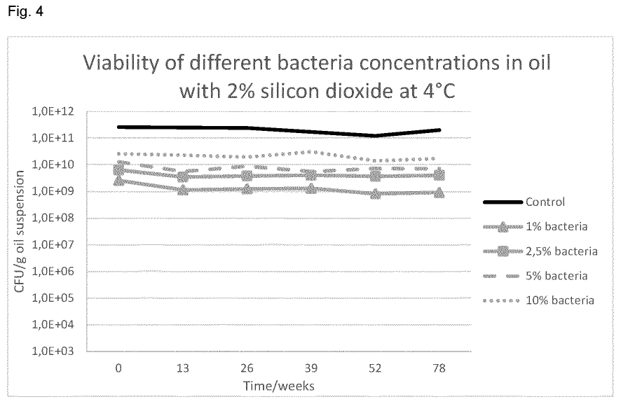

FIG. 3 shows that a blank lactobacilli count (before using the throat spray) of 5E01±2E01 CFU/2.5 cm² was found for all test persons and samples combined. This indicates that lactobacilli are present in the normal oral microbiome. An average retention after application of the probiotic throat spray formulation of 4E+05-8E05 CFU/2.5 cm² was obtained for all samples across all test persons after 30 minutes. This amount gradually decreased to 3E03-4E03 CFU/2.5 cm² after 2 hours and to 7E02-9E02 after 4 hours. The presence of lactobacilli in 5 out of 6 samples, in a count higher than 1E03 CFU/2.5 cm² is a successful outcome. With an application frequency of 3 to 5 times/day a protecting lactobacilli barrier can be obtained with the sprayable formulation for the duration of 12 to 24 hours.

Another successful result is that the recovered lactobacilli, after residing in the oral cavity for 4 hours, showed a clear inhibition zone versus the other endogenic present micro-organisms. This indicates that the robustness of the selected strains is an important parameter to take into account.

### Example 5—Throat Spray for Respiratory Virus Inhibition and Interferon Pathway Induction

### Material and Methods

### NF-kB and IRF Induction in THP1-Dual Monocytes

THP1-Dual monocytes (Invivogen) were maintained in RPMI 1640 (ThermoFisher Scientific) medium with 10% Fetal Calf Serum (FCS), 25 mM HEPES and 2 mM L-glutamine at 37° C., 5% CO2. For experiments with bacteria, THP1-Dual cells were seeded in a 96-well plate at a concentration of 10⁵ cells/well. Bacteria were added to the cells at 10⁶ CFU/well for live bacteria from cultures, 10⁷ CFU/well for UV inactivated bacteria from cultures, and 10⁸ CFU/well for powdered bacteria. Spray was added at a 1:20 dilution. The plate was incubated for 24 hours at 37° C. and 5% CO2. Induction of NF-κB was assessed based on SEAP reporter activity at 405 nm with the Synergy HTX Plate Reader (BioTek) after the addition of a para-Nitrophenylphosphate (pNPP) buffer. Induction of IRF was assessed based on luciferase reporter luminescence activity with the Synergy HTX Plate Reader (BioTek) after the addition of the QUANTI-Luc™ (InvivoGen) buffer. Poly(1:C) with Lipofectamine 2000 (Invitrogen) at 50 μg/ml for IRF induction or lipopolysaccharides (LPS) from E. coli (Sigma) at 20 ng/mL for NF-κB induction were used as positive controls.

### Spray Formulation and Assessment of Bacterial Viability

L. casei AMBR2, L. rhamnosus GG, and L. plantarum WCFS1 were formulated into an oral/throat targeting spray, based on a combination of bacterial powders in a sunflower oil suspension with Aerosil. The microbiome spray consisted of freeze-dried L. casei AMBR2, L. plantarum WCFS1, and L. rhamnosus GG, in a ratio of 50%, 33.3% and 16.7%, respectively.

Viability of the powders from the individual strains was assessed at 4° C. and 25° C. every 4 weeks over a time period of 6 months via resuspension of the powders in PBS and plating out serial dilutions on MRS agar. The amount of CFU/g powder was evaluated compared to the start concentration. For the mixture of the powders or the final spray formulation, viability was assessed at 4° C., 15° C. and 25° C. over a period of 6 months. Every 4 weeks, powders (after suspension in PBS) or spray were plated out in serial dilutions on MRS agar to measure the amount of CFU/g powder or spray.

### Evaluation of Lactobacilli Retention in the Throat of Healthy Participants

To evaluate whether the bacteria in the spray are able to temporary colonize the throat, 12 healthy male and female adult participants were asked to use the spray and collect swabs of the throat at the start, after 30 minutes, and after 2 hours of spray administration.

At each time point, 2 throat swabs were collected with eNATTM swabs for microbial DNA extraction using the PowerFecal DNA isolation kit (Qiagen), and in PBS for lactobacilli cultivation on MRS agar. Dual-index paired-end sequencing of the throat samples was performed on the V4 region of the 16S rRNA gene on a MiSeq Desktop sequencer (M00984, Illumina), as previously described (De Boeck et al. 2017, 2019).

For qPCR analysis, species-specific primers for L. casei AMBR2, L. plantarum 381 WCFS1 and L. rhamnosus GG were designed. Initially, a standard curve for each species was made to estimate the Ct-CFU ratio. The expression of the genes was quantified by RT-qPCR on a StepOne Plus Real-Time PCR System (v. 2.0; Applied Biosystems, Foster City, California, United States). Each DNA sample was amplified with PowerSYBR® Green PCR Master Mix (Applied Biosystems) in a total volume of 20 μL with 0.15 μM of each primer, 40 ng of cDNA and nuclease-free water. Throat swabs were collected by swabbing along the back of the throat and both tonsils and cultivated after resuspension in 1 ml PBS and plating out serial dilutions on MRS agar. Plates were incubated for 2 days at 37° C.

### Results

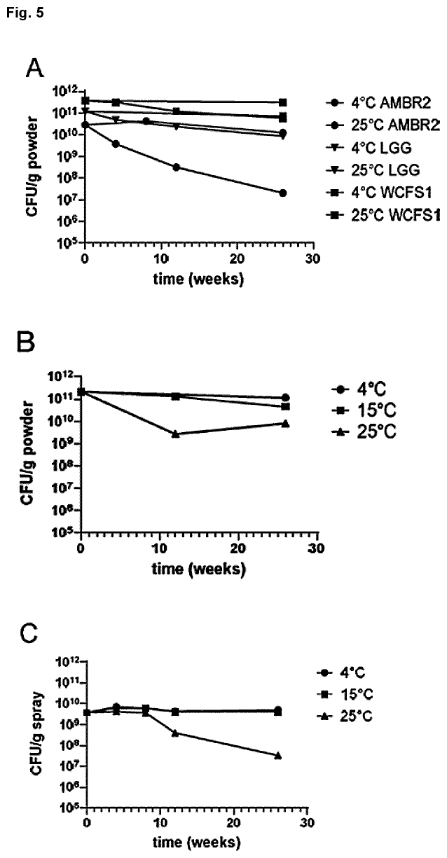

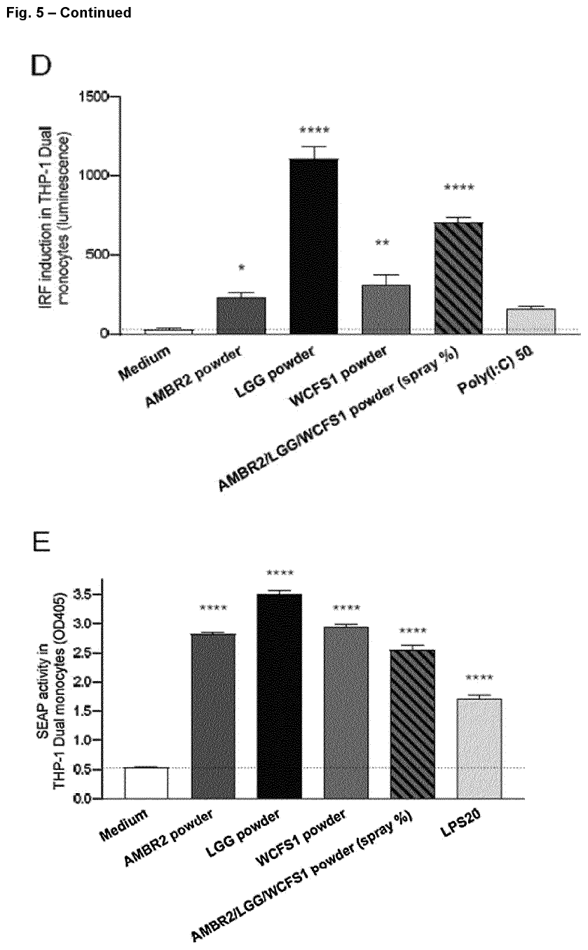

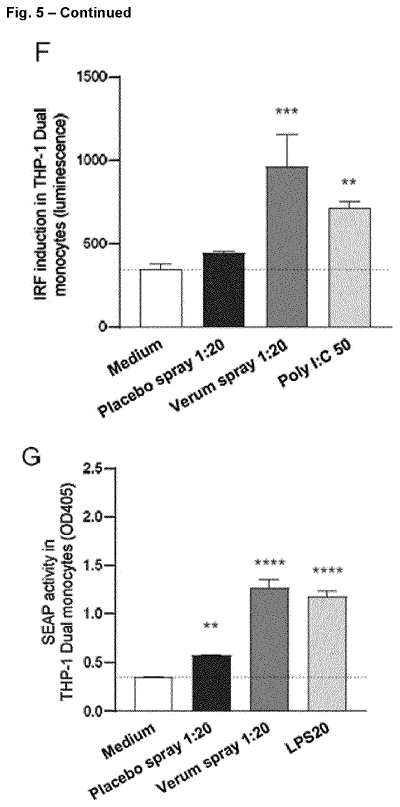

Although most probiotic sprays for the URT currently available consist of a bacterial suspension in saline or PBS, oil (as a non-volatile anhydrous carrier) was chosen to increase bacterial retention in the throat and to ensure a sufficient dosage of viable probiotic CFU counts. First, the viability of each bacterial strain in freeze-dried powder form was evaluated at 4° C. and 25° C. (FIG. 5A). For all three strains, the viability at 4° C. remained stable over time. For L. casei AMBR2 and L. plantarum WCFS1, no log reductions were observed, with 2.79×10¹⁰ CFU/g powder and 3.56×10¹¹ at start for AMBR2 and WCFS1, respectively, to 2.21×10¹⁰ CFU/g powder and 3.15×10¹¹ CFU/g powder after 26 weeks. For L. rhamnosus GG, the viability decreased from 1.18×10¹¹ CFU/g powder to 7.15×10¹⁰ CFU/g. At room temperature (25° C.), L. plantarum WCFS1 seemed most stable, with 1 log reduction at 26 week (5.84×10¹⁰), while this was 8.57×10⁹ for L. rhamnosus GG and 2.02×10⁷ for L. casei AMBR2.

We next evaluated different concentrations of the strains in the mixture. L. casei AMBR2 at 50%, L. plantarum WCFS1 at 33.3%, and L. rhamnosus GG at 16.7% were found the optimal ratio upon long term storage at room temperature, which reflected the intended storage conditions. Next, the viability of the combined bacterial strains in powder form (FIG. 5B) and in the throat spray formulation (FIG. 5C) was evaluated at 4° C, 15° C. and 25° C. For the mixed powders (FIG. 5B), viability decreased slightly from 2.09×10¹¹ CFU/g at the start, to 1.11×10¹¹, 4.51×10¹⁰ and 8.01×10⁹ CFU/g at 26 weeks of storage at 4° C., 15° C. and 25° C., respectively. For the spray formulation (FIG. 5C), viability starting with 3.78×10⁹ CFU/g spray remained stable at 4° C. and 15° C. at 26 weeks. At 25° C., a 2 log reduction was observed (3.3×10⁷ CFU/g) at 26 weeks.

We subsequently confirmed the retention of immunostimulatory activity in human monocytes of the strains and their combination in powder form (FIG. 5D-E), and in the spray formulation in oil (FIG. 5F-G). All single strains in powder form and their combination were still capable of significant IRF and NF-κB induction (FIG. 5D-E) at a dose of 10⁸ CFU/ml, which corresponds to the L. casei AMBR2 concentration per puff previously tested in healthy volunteers. The immunostimulatory action of the throat spray formulation with the three strains in an oil suspension was also compared to a placebo oil formulation without lactobacilli. The throat spray formulation with L. casei AMBR2 at 50%, L. plantarum WCFS1 at 33.3%, and L. rhamnosus GG at 16.7% in oil significantly induced IRF and NF-κB in human monocytes (FIG. 5F-G). While the placebo formulation also induced NF-κB, albeit to a lower degree compared to the spray formulation with lactobacilli, it did not significantly affect IRF.

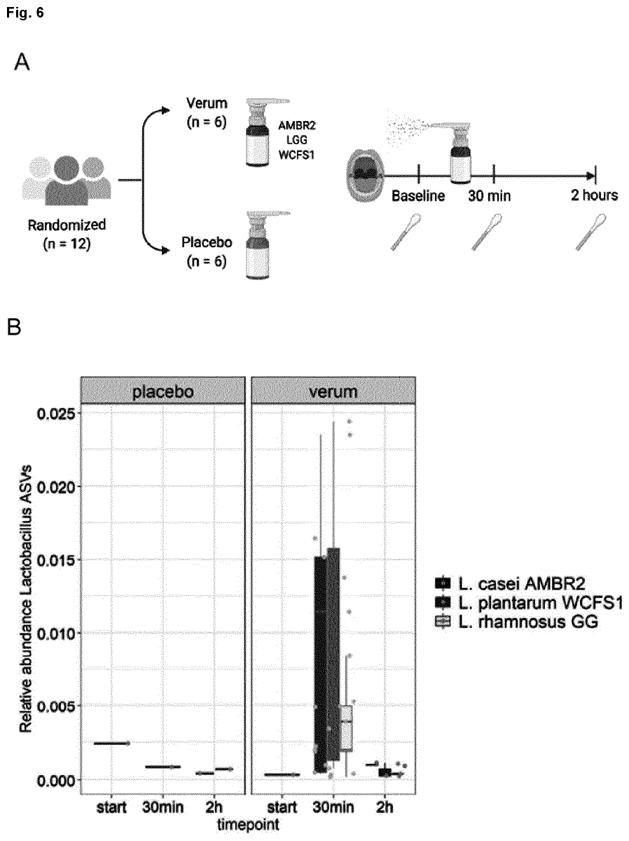

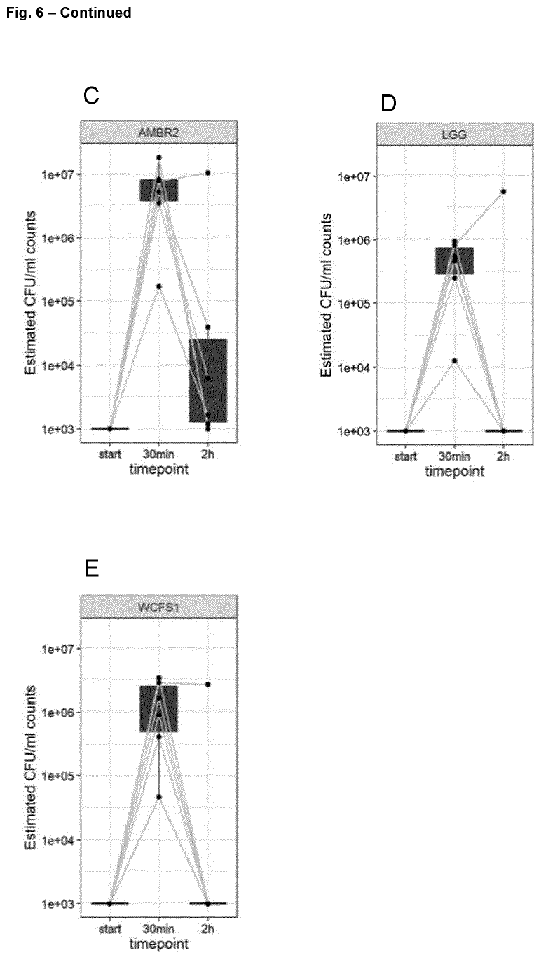

Finally, using a longitudinal placebo-controlled sampling set-up, we evaluated the retention of the lactobacilli administered in the formulated spray in the throat of 12 healthy volunteers. Presence of live bacteria was assessed via cultivation and quantitative polymerase chain reaction (qPCR), and overall retention of lactobacilli was quantified via 16S rRNA amplicon sequencing at DNA level (FIG. 6A). The volunteers used the verum spray at the start of the study by spraying two puffs containing approximately 9.5×10⁸ CFU of lactobacilli, or the placebo spray not containing lactobacilli, and throat swabs were collected at baseline, after 30 min, and after 2 hours (FIG. 6A). Microbiome analysis of the throat swabs showed that the dominant bacterial genera in the throat belong to canonical throat commensals, such as Prevotella, Veillonella, and Streptococcus species. A principal coordinate analysis (PCoA) plot based on the microbiome data across all time points did not reveal a clear clustering per treatment group at any of the tested time points (data not shown). A clear difference in relative abundances of Lactobacillaceae amplicon sequence variants (ASVs) was observed between the placebo and verum spray groups, especially 30 minutes after the spray was used (FIG. 6B). Three Lactobacillus ASVs, corresponding to the administered strains, were detected in the verum group 30 minutes after spray administration. Lactobacillus ASV 1 (L. rhamnosus) was detected in all 6 participants in the verum group, while Lactobacillus ASV 3 (L. casei) and Lactobacillus ASV 7 (L. plantarum) were detected in 5 out of 6 participants. In the placebo group, these Lactobacillus ASV were not detected, except for one participant that had low endogenous relative abundances of the Lactobacillus casei ASV after 30 minutes. After 2 hours, 5 out of 6 participants in the verum group still had detectable Lactobacillus ASVs.

To confirm and quantify the high abundances of the administered strains observed by sequencing DNA derived from samples in the verum group after bacterial administration, we aimed to estimate the CFU/ml counts based on targeted qPCR (FIG. 6C). In line with the sequencing data, after 30 minutes, the estimated CFU counts for L. rhamnosus GG in the verum group were between 1.26×10⁴-9.24×10⁵ CFU/ml. For L. casei AMBR2, estimated CFU/ml counts ranged from 1.72×10⁵ to 1.8×10⁷ CFU/ml and for L. plantarum WCFS1 from 4.63×10⁴ CFU-3.36×10⁶ CFU. After 2 hours, the amount of detected lactobacilli decreased. L. rhamnosus GG and L. plantarum WCFS1 were not detected anymore except in one participant. L. casei AMBR2 on the other hand, which was administered in the highest ratio of 50% in the spray, was still detected in 5 of the 6 participants, with a median CFU/ml count of 4×10³ CFU/ml.

In addition to analyzing the DNA of the bacteria, we also cultivated throat swabs to evaluate whether the administered lactobacilli were still viable. Cultured throat swabs from the verum group demonstrated colony morphologies typical for the three administered Lactobacilaceae strains, and the species identity was confirmed via colony PCR and sequencing of the 16S rRNA gene, confirming that the species corresponding to the ones administered with the spray are detected in the throat via their DNA, and can remain viable.

1. Sprayable formulation comprising powdered viable non-sporulating bacteria particles suspended in a non-volatile anhydrous liquid carrier, wherein at least 90% of the powdered bacteria particles have a particle size of less than 400 μm, wherein the sprayable formulation is under ambient pressure; and wherein said sprayable formulation is in the form of a liquid spray.

2. Sprayable formulation as claimed in claim 1, wherein the sprayable formulation does not comprise propellant gas, in particular carbohydrate propellant gas.

3. Sprayable formulation as claimed in claim 1 or 2, wherein the sprayable formulation sprayable liquid formulation is dispersed in droplets when sprayed.

4. Sprayable formulation as claimed in claim 1, wherein the non-volatile anhydrous liquid carrier has a vegetable origin, animal origin or mineral origin, preferably wherein the non-volatile anhydrous liquid carrier is an oily liquid carrier, more preferably wherein the non-volatile anhydrous liquid carrier comprises fatty acids, triglycerides, saturated or unsaturated fats, steroid derivatives or complex oils composing of phospholipids, sphingolipids, glycolipids or sulpholipids.

5. Sprayable formulation as claimed in any one of claims 1-4, wherein at least 90% of the powdered bacteria particles have a particle size of between 1 μm and 250 μm, preferably between 2 μm and 100 μm, more preferably between 5 and 50 μm, most preferably between 5 μm and 10 μm.

6. Sprayable formulation as claimed in any one of claims 1-5, wherein the concentration of powdered bacteria particles in the suspension is between 0.1-20 wt. %, preferably between 1-10 wt. %, most preferably between 3 and 7 wt. % in respect to the total weight of the sprayable formulation.

7. Sprayable formulation as claimed in any one of claims 1-6, wherein the formulation further comprises an anti-sedimentation agent.

8. Sprayable formulation as claimed in claim 7, wherein the anti-sedimentation agent is silicon dioxide or derivatives thereof.

9. Sprayable formulation as claimed in claim 7 or 8, wherein the concentration of the anti-sedimentation agent is 0.01-10 wt. %, preferably between 0.1-5 wt. %, more preferably between 0.5-3 wt. % and most preferably 1-2.5 wt. % in respect to the total weight of the sprayable formulation.

10. Sprayable formulation as claimed in any one of claims 1-9, wherein the sprayable formulation comprises between 50-99 wt. % non-volatile anhydrous liquid carrier, preferably between 70-98 wt. %, more preferably between 85 and 97 wt. %, most preferably between 90 and 95 wt. % in respect to the total weight of the sprayable formulation.

11. Sprayable formulation as claimed in any one of claims 1-10, wherein the sprayable formulation further comprises a rheological additive agent.

12. Sprayable formulation as claimed in any one of claims 1-11, wherein the sprayable formulation further comprises antioxidants and/or vitamins, in particular selected from vitamin D3 and E.

13. Sprayable formulation as claimed in any one of claims 1-12, wherein the formulation further comprises surfactants and/or emulsifiers and/or humectants.

14. Sprayable formulation as claimed in any one of claims 1-13, wherein the formulation further comprises bio-adhesives and/or mucoadhesives.

15. Sprayable formulation as claimed in any one of claims 1-14, wherein the formulation further comprises sweeteners and/or flavors.

16. Sprayable formulation as claimed in any one of claims 1-15, wherein the formulation further comprises formulation stabilizers in particular selected from the group consisting of epicatechins, quinones, creatin, hydroxytyrosol, pyridoxamine, cysteine, homocysteine, gluthation or other trapping alfa carbonyls.

17. Sprayable formulation as claimed in any one of claims 1-16, wherein the viable and/or stable bacteria are probiotic bacteria, in particular Lactobacillus species or Staphylococcus species.

18. Sprayable formulation as claimed in claim 17, wherein the Lactobacillus species are L. plantarum, L. pentosus, L. rhamnosus and/or L. casei.

19. Sprayable formulation as claimed in claim 1-18, wherein the sprayable formulation is an oronasopharyngeal spray.

20. Sprayable formulation as claimed in claim 1-18, wherein the sprayable formulation is a topical dermatological spray.

21. Sprayable formulation as claimed in any of claims 1-19 for use in human or veterinarian medicine.

22. Sprayable formulation as claimed in claim 1-21 for use in the prevention and/or treatment of viral, bacterial and/or fungal respiratory diseases.

23. Sprayable formulation as claimed in claim 1-21, for use in the prevention and/or treatment of coronaviral diseases.

24. Method for preparing the sprayable formulation as claimed in any one of claims 1-23, comprising the step of suspending powdered bacteria particles in an anhydrous carrier.

25. Method for enhancing the natural protective function of the oronasopharynx microbiome, comprising the step of applying the sprayable formulation as claimed in any one of claims 1-23.

26. Method for preventing and/or treating viral, bacterial and/or fungal respiratory diseases, comprising the step of applying the sprayable formulation as claimed in any one of claims 1-23.

27. Method for preventing and/or treating coronaviral diseases, comprising the step of applying the sprayable formulation as claimed in any one of claims 1-23.

28. Use of the sprayable formulation as claimed in any one of claims 1-23 for a topical dermatological application.

29. Use of the sprayable formulation as claimed in any one of claims 1-23 for cleaning surfaces.
